(19) United States
(12) Patent Application Publication
(10) Pub. No.: US 2008/0152717 A1
(43) Pub. Date: Jun. 26, 2008

(54) AMORPHOUS VALSARTAN AND THE PRODUCTION THEREOF

(75) Inventor: John A. Doney, Washington, DC (US)

Correspondence Address: INTERNATIONAL SPECIALTY PRODUCTS;WILLIAM J. DAVIS, ESQ., 1361 ALPS ROAD, WAYNE, NJ, 07470, US

(73) Assignee: ISP INVESTMENTS, INC., Wilmington, DE (US)

(21) Appl. No.: 11/955,527

(22) Filed: Dec. 13, 2007

Related U.S. Application Data
(60) Provisional application No. 60/874,885, filed on Dec. 14, 2006.

(51) Int. Cl. A61K 9/14; A61K 31/41; A61P 9/00

(52) U.S. Cl. 424/489; 514/381

(57) ABSTRACT

Valsartan compositions of enhanced bioavailability are described that contain valsartan with at least one solubility-enhancing polymer. Described methods to produce the bioenhanced products comprise solvent spray drying. One aspect of the method includes the steps of providing a mixture comprising valsartan, a solubility-enhancing polymer and a single solvent, a solvent blend or solvent/non-solvent blend removing and then evaporating the mixture to form amorphous valsartan.

### REFERENCE TO RELATED APPLICATION

This application claims the benefit of U.S. Provisional App. No. 60/874,885, filed Dec. 14, 2006, the contents of which are hereby incorporated by reference.

### BACKGROUND OF THE INVENTION

The present invention is directed to compositions containing amorphous valsartan and methods for producing amorphous valsartan. More particularly, the present invention relates to compositions and methods for preparing amorphous valsartan utilizing at least one solubility-enhancing polymer. In accordance with one embodiment, the valsartan is dissolved in a solvent containing the polymer. In yet another embodiment, a blend of solvent/non-solvent for the polymer is employed. The amorphous valsartan product can be produced by any method suitable to the composition. When necessary, solvent can be removed from compositions to yield the amorphous valsartan product. In one further development of the invention, a solution (or dispersion) comprising valsartan, polymer(s), and solvent(s) (or a solvent/non-solvent blend) is spray dried to produce valsartan in a form that exhibits improved bioavailability. The bioenhanced valsartan composition can be prepared by methods other than spray drying as recognized by those skilled in the art. Those methods include, without limitation: melt extrusion, spray congealing, granulation and freeze drying. In accordance with particular embodiments of the invention, a significant portion of the valsartan is provided in the amorphous state. In accordance with certain embodiments, the valsartan is converted almost entirely to the amorphous state. In one preferred embodiment of the invention, the valsartan is converted to the completely amorphous state.

Valsartan is known by the chemical name N-(1-oxopentyl)-N-[[2′-(1H-tetrazol-5-yl) [biphenyl]-4-yl]methyl]-L-valine. The empirical formula of valsartan is C₂₄H₂₉O₃ and its molecular weight is 435.5. Valsartan is a non-peptide, specific angiotensin II antagonist and is indicated for the treatment of hypertension. Valsartan is a white, crystalline powder that is insoluble in water.

Valsartan is sold commercially in conventional dosage forms and formulations as the branded drug, Diovan® (Novartis Pharmaceuticals Corp.) and is available in 40-, 80-, 160-, and 320-mg tablets. Excipients in these dosage forms include colloidal silicon dioxide, crospovidone, hydroxypropylmethylcellulose, magnesium stearate, iron oxides, microcrystalline cellulose, polyethylene glycol 8000, and titanium dioxide.

It is desirable to provide methods of producing valsartan exhibiting enhanced bioavailability compared to the crystalline form of the compound. It is generally well known by those in the field that crystalline valsartan exhibits low aqueous solubility and a negative food effect (i.e., drug plasma concentrations decrease when the drug is administered with food). By converting a substantial portion of crystalline valsartan to the amorphous state, these constraints that limit bioavailability are diminished. Furthermore, valsartan presented as an amorphous solid may facilitate manufacturing of both the active ingredient and the finished product and enable the use of reduced size dosage forms. Moreover, the selective customization of the properties of particles comprising valsartan can offer intriguing opportunities for pharmaceutical production and drug delivery. The morphology of individual particles plays a central role in this pursuit, since morphology directly influences bulk powder properties, such as density, residual solvent content, and flowability. In addition, techniques that modify particle shape and interior structure may profoundly affect pharmacokinetic properties, such as drug release rate, solubility, and bioavailability. Thus, the ability to design particle morphology has significant implications for the production process and product attributes.

### SUMMARY OF THE INVENTION

The present invention provides compositions and methods for producing amorphous valsartan. A composition comprising a solid dispersion of valsartan and at least one solubility-enhancing polymer wherein the valsartan in the dispersion is substantially amorphous is provided in certain embodiments of the present invention. In one aspect, the disclosed invention describes the conversion of crystalline valsartan to the amorphous state. One method for producing this conversion is through solvent spray drying. Other techniques that accomplish this conversion include, without limitation: flash solvent evaporation, melt-congeal spraying, freeze drying, and melt-extrusion. These methods can use a single solubility-enhancing polymer or blends of polymers. The degree of valsartan amorphous conversion depends on various factors, including, but not limited to, polymer type and amount and processing conditions. In accordance with certain aspects of the invention, a single organic solvent, blends of solvents, or solvent/non-solvent blends can be used.

In one aspect, the invention relates to spray-dried powders or granulated products comprising amorphous valsartan. In addition, the resulting powders produced in accordance with certain embodiments typically possess lower residual solvent content and higher tap density than their counterparts produced by conventional methods, due to a change in the particle morphology and size. When applied to produce pharmaceutical products, a system of polymers can be used to modify not only particle morphology, but also the pharmacokinetic properties of the active.

One aspect of the invention involves amorphous valsartan prepared from compositions containing valsartan and a solubility-enhancing polymer in a solvent or solvent blend. This solvent or solvent blend includes one or more solvents in which the polymer is soluble. The term “soluble” means that the attractive force between polymer and solvent molecules is greater than the competing inter- and intramolecular attractive forces between polymer molecules. For simplicity, this solvent is simply called “solvent.” Compositions also are described in which the solvent blend contains a solvent for which the opposite is true: The attractive force between polymer and solvent molecules is less than the inter- and intramolecular attractive force between polymer molecules. This second solvent is termed the “non-solvent.” The polymer may swell but does not dissolve in the non-solvent. In accordance with one embodiment of the invention, a solubility-enhancing polymer and a suitable solvent/non-solvent blend are provided. Additionally, the solvent possesses a lower boiling point than the non-solvent. Preferably, the solvent and non-solvent are miscible. The ratio of solvent to non-solvent is such that the polymer can be considered “dissolved” in the solvent system.

Unique particle properties can be created by evaporating the solvent/non-solvent blend. For example, this evaporation can occur during the spray drying of the feed solution or granulation processes. Atomized droplets containing a blend of solvents will experience a change in the total solvent composition due to evaporation. The method appears to be independent of how the droplets are generated or atomized. Initially, the polymer exists in a dissolved state, due to a sufficient amount of the solvent. As it evaporates (the solvent boils at a lower temperature than the non-solvent), the concentration of non-solvent in the droplet increases. Eventually, the solvent composition is insufficient to maintain the polymer in solution. In doing so, the polymer collapses from solution. This change in polymer conformation can alter the evaporation dynamics of the droplet to create particle morphologies that influence final powder properties.

The use of a solvent/non-solvent blend system has been found to provide additional benefits beyond the benefits obtained with a solvent only system. This solvent/non-solvent approach can produce a spray dried powder of lower residual solvent content and smaller particle size. A further consequence of this engineered particle morphology is the increase in bulk powder density. Increased powder density is an important attribute for many applications. The extent of polymer collapse—and therefore the net effect on the spray dried powder properties—depends on the polymer salvation factors, such as the initial ratio of solvent to non-solvent, the polymer chemical structure and the polymer molecular weight. In addition to reducing residual solvent content and increasing density, the primary polymer may be paired with the solvent/non-solvent system in order to affect not only the morphology of the particle, but also that of the valsartan, and thereby affect the valsartan loading, crystallinity, solubility, stability and release.

The presence of additional polymers may contribute to the final particle morphology by their interaction with the first polymer and the solvent system. These additional polymers may also be advantageous to create special release properties of the active. For example, the primary polymer may be paired with the solvent/non-solvent system in order to affect particle morphology, and thereby residual solvent content and bulk powder density. Additional polymeric adjuvants may be added to serve additional purposes: further inhibit active recrystallization, further maximize active concentration, and further enhance/delay/retard dissolution rate. To accomplish these functionalities, it is necessary to suitably match the adjuvant solubilities with the solvent blend selected for the primary polymer.

### BRIEF DESCRIPTION OF THE DRAWINGS

FIG. 1 is a plot of heat flow versus temperature for spray dried samples produced in accordance with Example 1.

FIG. 2 is a plot of percent valsartan released in water as a function of time for spray dried product using pvp and the solvent only method in accordance with Example 1.

FIG. 3 is a plot of percent valsartan released in water as a function of time for spray dried product using hpc and the solvent/non-solvent method in accordance with Example 1.

### DETAILED DESCRIPTION OF THE INVENTION

The term “comprising” encompasses the more restrictive terms “consisting essentially of” and “consisting of.”

All percentages, ratios and proportions used herein are by weight unless otherwise specified.

The term “solid dispersion” as used herein refers to a system in a solid state comprising at least two components, wherein one component is dispersed evenly throughout the other component or components. The term “solid dispersion” includes systems having small particles either completely crystalline, completely amorphous or any state in between, typically less than about 1 μm in diameter, of one phase dispersed in another phase.

The term “solid solution” as used herein refers to a type of solid dispersion wherein one component is molecularly dispersed throughout another component such that the system is chemically and physically uniform and homogeneous throughout. These systems do not contain any significant amounts of active ingredients in their crystalline or microcrystalline state as evidenced by thermal analysis or x-ray diffraction.

The term “solubility-enhancing polymer” refers to a polymer that provides at least one of the following properties as a result of its presence in the composition compared to a control composition without the solubility-enhancing polymer:
a) an increase in initial release of at least about 25%
b) an increase in extent of release of at least about 25%
c) an increase in maximum plasma concentration of at least about 25%
d) an increase in AUC₀₋₂₄ₕ of at least about 25%.

Although the following description is primarily directed to the preparation of a spray-dried composition containing valsartan, the present invention is not limited to valsartan spray-dried compositions. The scope of the invention includes other methods described herein that are also useful in converting valsartan to the amorphous state and corresponding enhanced bioavailability. Those methods include, without limitation: melt extrusion, spray congealing, granulation and freeze drying.

There is no condition placed on the state of the compositions other than amorphous valsartan combined with one or more solubility-enhancing polymer(s). The term “combined” includes, but is not limited to: blended, co-mingled, dissolved, extruded, granulated, melted, milled, mixed, sieved, slurried, sprayed, stirred, and the combination of these and other methods. Other techniques may be identified by those skilled in the art.

In accordance with one embodiment, the present invention is related to a method for preparing a spray-dried composition by providing a mixture containing valsartan and a polymer in a single solvent, a solvent blend or a blend of a solvent and a non-solvent for the polymer and spray drying the mixture to form the spray-dried composition.

In accordance with one aspect of the invention, a polymer system is provided comprising a polymer—called the primary polymer—and a suitable solvent or solvent blend. This approach comprises a solvent in which the polymer is soluble. Guidance in defining polymer solubility is provided by the expansion coefficient (α):

$$α = \frac{{( \overline{r} )}^{1 / 2}}{{( {\overline{r}}_{o}^{2} )}^{1 / 2}} \qquad ( § 1 )$$

where r̅² is the mean-square distance between chain ends, and r̅ₒ² is the unperturbed dimension. (Equation §1 can be written for branched polymers in an analogous manner, using square-average radius of gyration about the center of gravity, s̅², and the corresponding unperturbed dimension, s̅ₒ².) Polymer solubility is provided when α is unity or greater, and solvents that satisfy this condition are called “good solvents,” or simply “solvents.” Solvents uncoil (or expand) the polymer molecule, since the polymer-solvent attractive force is greater than that of polymer-polymer. Light scattering methods, such light scattering detectors (e.g., Triple Detector Array, Viscotek Corp.), can be used to determine the variables expressed in equation §1. These concepts are defined in the text Polymer Chemistry, An Introduction, by Malcolm P. Stevens, which is incorporated by reference.

When α equals unity, a special condition exists in that polymer-solvent and polymer-polymer forces are balanced. Solvents that enable this condition are called θ solvents. Within the context of this invention, solvents are considered “good solvents” when α is about equal to 1 or more. It is appreciated that temperature influences α, such that a good solvent may be transformed into a non-solvent merely by changing the temperature.

In yet another embodiment of this invention, the solvent blend also contains a solvent for which the opposite is true: Polymer-polymer forces dominate polymer-solvent forces. In this case, α is less than one and the solvent is termed a “non-solvent,” because the polymer exists in a collapsed state. In accordance with one embodiment of the invention, one polymer is provided with a suitable solvent/non-solvent blend. The blend of solvent/non-solvent maintains a solvated state of the polymer, such that the polymer can be considered “dissolved” in the solvent system. Additionally, the solvent possesses a lower boiling point than the non-solvent. (Solvent/non-solvent pairs that form an azeotrope do not satisfy this criterion.) Preferably, the solvent and non-solvent are miscible.

Unique particle properties can be created by evaporating the solvent/non-solvent blend. For example, this evaporation can occur during the spray drying of the feed solution or granulation processes. Atomized droplets containing a blend of solvents will experience a change in the total solvent composition due to evaporation. The method appears to be independent of how the droplets are generated or atomized. Initially, the polymer exists in a dissolved state, due to a sufficient amount of the solvent. As it evaporates (the solvent boils at a lower temperature than the non-solvent), the concentration of non-solvent in the droplet increases. Eventually, the solvent composition is insufficient to maintain the polymer in solution. In doing so, the polymer collapses and precipitates from solution. This change in polymer conformation can alter the evaporation dynamics of the droplet to create particle morphologies that influence final powder properties. Examples of suitable polymer/solvent/non-solvent combinations include, without limitation, polyvinylpyrrolidone/dichloromethane/acetone, polyvinylpyrrolidone-co-vinyl acetate/acetone/hexane, and ethylcellulose/acetone/water.

Unique particle architectures are created by precipitation of the polymer when the non-solvent concentration exceeds a critical value. This critical ratio R_{c} can be defined:

$${R}_{c} = \frac{mass nonsolvent}{mass solvent + nonsolvent } ,$$

which is the maximum fraction of the non-solvent before polymer collapse occurs. The ratio R_{c} for a given system can be determined experimentally by identifying the mass fractions of each component that produce a significant increase in solution turbidity. If an R_{c} value can be identified for a system, then the system comprises a solvent/non-solvent blend. One example is a solution consisting of about 10% (w/w) polyvinylpyrrolidone, 18% (w/w) dichloromethane, and 72% (w/w) acetone, for which R_{c} equals 0.80. Polymer systems will typically be used at solvent/non-solvent blends that are below the R_{c} value for the system. It may be advantageous to formulate more complex polymer/solvent systems in order to control particle morphology/size as well as the crystallinity, solubility, bioavailability and/or release characteristics of the valsartan.

The present invention in accordance with other embodiments provides a method to increase the density of spray-dried powders. Typically, spray drying produces sphere-like particles with some degree of interior void. This void increases particle bulk without mass and creates low-density material. Adding a non-solvent to the working solution/dispersion changes the particle size and morphology, leading to an increase in density. Particles may be smaller, wrinkled, dimpled, and/or collapsed compared to those prepared using only solvent. The solvent/non-solvent approach also reduces the mean particle size, allowing the powder to pack better. In addition, powder flow and powder-powder mixing properties are enhanced.

The present invention in accordance with certain aspects provides a method to reduce or eliminate the need for secondary drying of spray-dried powders and granulated materials. These products often contain residual solvent, and it is desirable or necessary to produce a drier product. A high residual solvent content can result from formulation or processing limitations. The general practice has been to use a solvent that dissolves the solids being spray dried. In doing so, solvent can be trapped inside the spray dried powder or granulated bead due to case hardening. The intentional pairing of a lower-boiling solvent with a higher-boiling non-solvent for the materials being processed can yield products of lower residual solvent due to the effect(s) of the non-solvent on the process polymers.

The present invention may further provide a method to enhance the aqueous solubility, modify the release of active ingredients through selection of a polymer system with the solvent or solvent/non-solvent blend, and thereby diminish the constraints that limit valsartan bioavailability. The polymer system is chosen so that one (or more) polymer(s) work with the solvent/non-solvents to create novel particle morphologies. Additional polymer(s) may be added as needed to affect the solubility and release properties of the valsartan, as well as particle morphology. Enhanced solubility can be achieved by a number of factors, including (but not limited to): improved wettability, creation of amorphous drug forms, stabilization against recrystallization, and/or co-solvation effects. In doing so, a supersaturated solution of the valsartan is produced. “Modified release” refers to changing the time frame in which the active is released, i.e., immediate, delay, extended. These modified releases are created by matching functional polymer(s) with the appropriate solvent/non-solvent blend.

Solvents and non-solvents suitable for use in the process of the present invention can be any organic compound (including water) in which the primary polymer is soluble in the case of solvents, or insoluble, in the case of non-solvents. The choice and ratio of solvent/non-solvent depends on the choice of the primary polymer. Accordingly, the identification of an organic compound as a solvent or non-solvent depends on the primary polymer. Therefore, a solvent in one system may be a non-solvent in another. Particularly useful solvents and non-solvents include, but are not limited to: acetic acid, acetone, acetonitrile, anisole, 1-butanol, 2-butanol, butyl acetate, tert-butylmethyl ether, chlorobenzene, chloroform, cumene, cyclohexane, 1-2-dichloroethane, dichloromethane, 1-2-dimethoxyethane, N—N-dimethylacetamide, N—N-dimethylformamide, 1-4-dioxane, ethanol, 2-ethoxyethanol, ethyl acetate, ethylene glycol, ethyl ether, ethyl formate, formamide, formic acid, heptane, hexane, isobutyl acetate, isopropyl acetate, methanol, methyl acetate, 2-methoxyethanol, 3-methyl-1-butanol, methylbutylketone, methylcyclohexane, methylethyl ketone, methylisobutyl ketone, 2-methyl-1-propanol, N-methylpyrollidone, nitromethane, pentane, 1-pentanol, 1-propanol, 2-propanol, propyl acetate, pyridine, sulfolane, tetrahydrofuran, tetralin, 1-2-2-trichloroethene, toluene, water, and xylene. Mixtures of solvents and mixtures of non-solvents can also be used. In accordance with particular embodiments, solvent blends at the azeotropic composition (which boil at one common temperature) can comprise either the solvent or non-solvent, but not the solvent/non-solvent blend.

Solubility-enhancing polymers that enhance bioavailability suitable for use in the mixtures of the present invention should result in conversion of at least some of the crystalline valsartan to the amorphous state. In accordance with those embodiments wherein a solvent/non-solvent blend is used, at least one polymer should be soluble in the solvent and not soluble in the non-solvent. Specific examples of useful polymers include, but are not limited to: aliphatic polyesters (e.g., poly D-lactide), carbohydrates (e.g., sucrose), carboxyalkylcelluloses (e.g., carboxymethylcellulose), alkylcelluloses (e.g., ethylcellulose), gelatins, hydroxyalkylcelluloses (e.g., hydroxymethylcellulose), hydroxyalkylalkylcelluloses (e.g., hydroxyethylmethyl cellulose), hydroxyalkylalkylcellulose derivatives (e.g. hydroxypropylmethyl cellulose phthalate, hydroxypropylmethyl cellulose acetate succinate) polyamines (e.g., chitosan), polyethylene glycols (e.g., PEG 8000, PEG 20000), methacrylic acid polymers and copolymers (e.g., Eudragit series of polymers), homo- and copolymers of N-vinyl pyrrolidone (e.g., polyvinylpyrrolidone, polyvinylpyrrolidone-co-vinyl acetate), homo- and copolymers of vinyllactam, starches, polysaccharides (e.g., alginic acid), poly glycols (e.g., propylene glycol, polyethylene glycol), polyvinyl esters (e.g., polyvinyl acetate), and refined/modified shellac. The amount of the polymer present in the mixture may range from about 1% to about 95%, more particularly from about 5% to 90%, by weight of the mixture, and in accordance with certain embodiments from about 25% to 75% by weight. Blends of polymers may also be used.

The spray-dried mixture includes valsartan as an active ingredient. The mixture may contain from about 1% to about 95% active, more particularly from about 20% to about 80% active, depending on the desired dose of the active. The weight ratio of valsartan to polymer typically will be from about 95% valsartan:5% total polymer to about 5% valsartan:95% total polymer, more particularly from about 70% valsartan:30% total polymer to about 30% valsartan:70% total polymer and in accordance with certain aspects from about 60% valsartan:40% total polymer to about 40% valsartan:60% total polymer.

The compositions of the present invention produce composition wherein at least a portion of valsartan is in the amorphous state. The term “amorphous” refers to a compound in a non-crystalline state. In other words, an amorphous compound lacks long-ranged, defined crystalline structure. In accordance with certain embodiments of the present invention, at least some, more particularly at least about 10%, at least about 25%, or at least about 40% of the valsartan in the composition is in an amorphous form. In other embodiments, at least a major portion of the compound in the composition is amorphous. As used herein, the term “a major portion” of the compound means that at least about 50% of the compound in the composition is in the amorphous form, rather than the crystalline form. More particularly, the compound in the composition may be substantially amorphous. As used herein, “substantially amorphous” means that the amount of the compound in the crystalline form does not exceed about 25% (i.e., more than about 75% of the compound is in the amorphous form). In accordance with particular embodiments of the invention, the compound in the composition is “almost completely amorphous” meaning that the amount of drug in the crystalline form does not exceed about 10% (i.e., more than about 90% of the compound is in the amorphous form). Compositions are also provided wherein the compound in the composition is considered to be “completely amorphous” meaning that the crystalline form of the drug is not detectable using conventional techniques, such as x-ray diffraction or thermal analysis. Reference to a composition as completely amorphous does not exclude compositions containing trace amounts (less than about 1%) of the crystalline form of the drug.

Amorphous materials lack some measurable properties, such as melting endotherms as measured by differential scanning calorimetry that characterize crystalline forms. Amounts of crystalline drug may be measured by powder x-ray diffraction (PXRD), differential scanning calorimetry (DSC), or any other standard quantitative analysis. The amounts of crystalline valsartan present in the composition may be detected by other standard measurement known to those of ordinary skill in the art. It is appreciated that the measurement of such properties may be dependent on instrument type and sensitivity.

By providing the valsartan in the amorphous form, the spray dried product produced in accordance with the present invention provides enhanced bioavailability of VAL compared to products containing the principle crystalline form. The increased bioavailability of the active can also lead to reduced dosage sizes and dose amounts for the active. Applicants have also determined that the rate of drug release can be controlled through proper selection of the polymers added into the solvent solution for processing. In certain embodiments of the invention, the process is spray drying.

The spray dried mixture may also contain additional polymeric materials that can modify properties of the composition. For example, certain polymers can be included to control particle morphology/size as well as the bioavailability and release characteristics of the active ingredient. Additional polymers may also be included in the mixture to further inhibit active recrystallization, further maximize active concentration and further enhance/delay/retard dissolution rate. Additional polymers that can be incorporated into this system are not particularly limited.

The mixture to be spray dried typically contains from about 40% to 99.9% by weight total solvent or solvent/non-solvent, more particularly from about 80% to 95% by weight total solvent or solvent/non-solvent based on the total weight of the mixture. When a solvent/non-solvent blend is used, the critical ratio R_{c} can vary from about 0.01-0.99, more particularly from about 0.1-0.9, still more particularly from about 0.3-0.8.

In addition to the solvent, polymer and valsartan, the mixture to be spray dried may also include other ingredients to improve performance, handling or processing of the mixture. Alternatively, these ingredients also may be admixed into the already-prepared valsartan-polymer by methods including, but not limited to tumble blending and granulation technologies. Typical ingredients include, but are not limited to, silica, surfactants, pH modifiers, fillers, complexing agents, solubilizer, pigments, lubricants, glidants, flavor agents, plasticizers, taste masking agents, etc.

The spray drying apparatus used in the process of the present invention can be any of the various commercially available apparatus. Examples of specific spray drying devices include spray dryers manufactured by Niro Inc. (e.g., SD-Micro®, PSD®-1, PSD®-2, etc.), the Mini Spray Dryer (Buchi Labortechnik AG), spray dryers manufactured by Spray Drying Systems, Inc. (e.g., models 30, 48, 72), and SSP Pvt. Ltd.

Spray drying processes and spray drying equipment are described generally in Perry's Chemical Engineers' Handbook, Sixth Edition (R. H. Perry, D. W. Green, J. O. Maloney, eds.) McGraw-Hill Book Co. 1984, pages 20-54 to 20-57. More details on spray drying processes and equipment are reviewed by Marshall “Atomization and Spray Drying,” 50 Chem. Eng. Prog. Monogr. Series 2 (1954). The contents of these references are hereby incorporated by reference.

The term “spray drying” is used conventionally and, in general, refers to processes involving breaking up liquid mixtures into small droplets and rapidly removing solvent from the mixture in a container (spray drying apparatus) where there is a strong driving force for evaporation of solvent from the droplets. Atomization techniques include two-fluid and pressure nozzles, and rotary atomizers. The strong driving force for solvent evaporation is generally provided by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas; or (3) both.

Generally, the temperature and flow rate of the drying gas and spray dryer design are chosen so that the polymer/active solution droplets are dry enough by the time they reach the wall of the apparatus that they are essentially solid and so that they form a fine powder and do not stick to the apparatus wall. It is also possible to operate a spray dryer so that product collects on the apparatus wall, and then is collected by removing the material manually, pneumatically, mechanically or other means. The actual length of time to achieve the preferred level of dryness depends on the size of the droplets, the formulation, and spray dryer operation. Following the solidification, the solid powder may stay in the spray drying chamber for 5-60 seconds, further evaporating solvent from the solid powder. The final solvent content of the solid dispersion as it exits the dryer should be low, since this improves the stability of the product. Generally, the residual solvent content of the spray-dried composition should be less than about 10% by weight and preferably less than about 2% by weight. In accordance with certain embodiments, the residual solvent content is within the limits set forth in the International Conference on Harmonization (ICH) Guidelines. Although not typically required in accordance with certain aspects of the present invention, because the presence of a non-solvent produces a spray-dried powder of lower residual solvent content, it may be useful in accordance with certain embodiments of the present invention to subject the spray-dried composition to further drying to lower the residual solvent to even lower levels. Methods to further lower solvent levels include, but are not limited to fluid bed drying, infra-red drying, tumble drying, vacuum drying, and combinations of these and other processes. Additional detail with respect to a particular spray drying process is described in more detail in the examples. However, the operating conditions to spray dry a powder are well known in the art and can be easily adjusted by the skilled artisan. Furthermore, the examples describe results obtained with a laboratory scale spray dryer. One of ordinary skill in the art would readily appreciate the variables that must be modified to obtain similar results with a production scale unit.

As indicated above, the present invention is not limited to amorphous valsartan produced by spray drying. In addition to spray drying, compositions of the present invention may be prepared by other processes including, but not limited to, extrusion, spheronization and spray congealing.

Extrusion is a well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders.

In melt extrusion, components can be melted and extruded with a continuous process with or without solvent and with or without inclusion of additives. Such a process is well-established and well-known to skilled practitioners in the art.

Spheronization is the process of converting material into spheres, the shape with the lowest surface area to volume ratio. Spheronization typically begins with damp extruded particles. The extruded particles are broken into uniform lengths instantaneously and gradually transformed into spherical shapes. In addition, powdered raw materials, which require addition of either liquid or material from a mixer, can be processed in an air-assisted spheronizer.

Spray congealing is a method that is generally used in changing the structure of the materials, to obtain free flowing powders from liquids and to provide pellets ranging in size from about 0.25 to 2.0 mm. Spray congealing involves allowing a substance of interest to melt, disperse, or dissolve in a hot melt of other additives. The molten mixture is then sprayed into an air chamber wherein the temperature is below the melting point of the formulation components, to provide spherical congealed pellets. The temperature of the cooled air used depends on the freezing point of the product. The particles are held together by solid bonds formed from the congealed melts. Due to the absence of solvent evaporation in most spray congealing processes, the particles are generally non porous and strong, and remain intact upon agitation. The characteristics of the final congealed product depend in part on the properties of the additives used. The feed rate and inlet/outlet temperatures are adjusted to ensure congealing of the atomized liquid droplet. The feed should have adequate viscosity to ensure homogeneity. The conversion of molten feed into powder is a single, continuous step. Proper atomization and a controlled cooling rate are critical to obtain high surface area, uniform and homogeneous congealed pellets. Adjustment of these parameters is readily achieved by one skilled in the art.

The spray congealing method is similar to spray drying, except that solvent is not used. Instead, the active ingredient(s) is dispersed and/or melted into a matrix comprising melt-processable polymer(s). Spray congealing is a uniform and rapid process, and is completed before the product comes in contact with any equipment surface. Most actives and additives that are solid at room temperature and melt without decomposition are suitable for this method.

Conventional spray dryers operating with cool inlet air have been used for spray congealing. Several methods of atomization of molten mass can be employed, such as pressure, or pneumatic or centrifugal atomization. For persons skilled in the spray congealing art, it is well known that several formulation aspects, such as matrix materials, viscosity, and processing factors, such as temperature, atomization and cooling rate affect the quality (morphology, particle size distribution, polymorphism and dissolution characteristics) of spray congealed pellets. The spray congealed particles may be used in tablet granulation form, encapsulation form, or can be incorporated into a liquid suspension form.

Valsartan produced in accordance with some embodiments of the invention exhibit enhanced bioavailability when present in solid state forms such as solid solutions or solid dispersions. The valsartan may be present in such compositions at levels exceeding about 5% by weight, more particularly exceeding about 10%, and in some cases exceeding about 25%, 40%, 50% or even 75% by weight of the composition and still exhibit enhanced bioavailability compared to crystalline forms of the compound.

Certain polymers function as solubility-enhancing polymers in that the presence of the polymer in the composition improves solubility of the valsartan under various conditions. The solubility-enhancing polymer provides at least one of the following properties as a result of its presence in the composition compared to a control composition without the solubility-enhancing polymer or to a composition containing the crystalline form of the drug:
a) an increase in initial release of at least about 25%, more particularly at least about 100% and in accordance with certain embodiments at least about 200%
b) an increase in extent of release of at least about 25%, more particularly at least about 100% and in accordance with certain embodiments at least about 200%
c) an increase in maximum plasma concentration of at least about 25%, more particularly at least about 100% and in accordance with certain embodiments at least about 200%
d) an increase in AUC₀₋₂₄ₕ of at least about 25%, more particularly at least about 100% and in accordance with certain embodiments at least about 200%.

Initial release refers to the percent of drug released after 15 minutes in accordance with a standard dissolution test method. Extent of release refers to the percent of drug released after 75 minutes in accordance with the same standard dissolution test method.

In accordance with particular embodiments of the present invention, a composition prepared from a system comprising a polymer and an valsartan spray dried from a solvent/non-solvent system as described herein exhibits a dissolution profile wherein the percent active released at some point in time is at least about 25%, more particularly at least about 50% and in certain cases at least about 100% greater than a control composition prepared from a system comprising the same polymer and valsartan spray dried from the same solvent without the non-solvent. Preferably these limits are obtained within about 120 minutes, more particularly within about 60 minutes and still more particularly within about 30 minutes. Dissolution profiles can be determined using USP apparatus II (paddles) (VK 7010®, Varian Inc.), with a bath temperature of 37° C. and a paddle speed of 100 rpm for 60 minutes.

In accordance with particular embodiments of the present invention, a composition prepared from a system comprising a polymer and an valsartan spray dried from a solvent/non-solvent system as described herein exhibits an increase in bulk density or tap density wherein the density is at least about 25%, more particularly at least about 50% and in certain cases at least about 100% greater than a control composition prepared from a system comprising the same polymer and valsartan spray dried from the same solvent without the non-solvent.

Valsartan compositions prepared from a solvent/non-solvent system typically result in reduced particle size. In accordance with particular embodiments of the present invention, a composition prepared from a system comprising a polymer and valsartan spray dried from a solvent/non-solvent system as described herein results in a reduction of particle size on the order of at least about 50%, more particularly at least about 100% and in certain cases at least about 300% compared to a control composition prepared from a system comprising the same polymer and valsartan spray dried under similar conditions from the same solvent without the non-solvent.

The compositions and methods described herein to produce bioenhanced valsartan product may improve one or more pharmacokinetic parameters relative to the crystalline form of the drug. This invention may increase drug exposure, as measured by the area under the drug plasma concentration vs. time curve (AUC), an effect that may be attributed to the enhanced aqueous solubility. Additionally, the enhanced bioavailability may reflect a diminished food effect; food decreases the AUC and maximum drug plasma concentration (Cₘₐₓ) of products containing crystalline valsartan. In yet another embodiment of the invention, enhanced valsartan bioavailability is provided by reduced inter-patient variability of these pharmacokinetic parameters.

Compositions of the present invention may be delivered by a wide variety of routes, including, but not limited to: buccal, dermal, intravenous, nasal, oral, pulmonary, rectal, subcutaneous, sublingual, and vaginal. Generally, the oral route is preferred.

Compositions of the invention may be presented in a numerous forms. Exemplary presentation forms are powders, granules, and multiparticulates. These forms may be added directly to capsules; compressed to produce tablets, capsules, or pills; or reconstituted by addition of water or other liquids to form a paste, slurry, ointment, suspension or solution. Various additives may be mixed, ground, or granulated with the compositions of this invention to form a material suitable for the above dosage forms.

Compositions of the invention may be formulated in various forms so that they are delivered as a suspension of particles in a liquid vehicle. Such suspensions may be formulated as a liquid or as a paste at the time of manufacture, or they may be formulated as a dry powder with a liquid, typically water, added at a later time but prior to oral administration. Such powders that are constituted into a suspension are often referred to as sachets or oral powders for constitution (OPC). Such dosage forms can be formulated and reconstituted via any known procedure.

Oral, solid-dose pharmaceutical spray dried powders typically have a mean particle size of about 0.5 μm-500 μm and are generally prepared from solutions at concentrations of 1% or more total solids, more particularly from about 2%-50%, and still more particularly from about 3%-30% solids.

Oral, solid dose pharmaceutical granules typically have a mean particle size of about 50 μm-5000 μm. Techniques to produce granules include, but are not limited to, wet granulation and various fluid bed granulating methods.

Pharmaceutical compositions comprising the valsartan of enhanced bioavailability described herein may be prepared in accordance with conventional techniques. In accordance with one aspect of the invention, a pharmaceutical dosage form is provided comprising valsartan and a disintegrant. The disintegrant used in the composition is preferably of the so-called superdisintegrant type, disintegrants of this type being well-known to the person skilled in the art. As examples of these disintegrants the following can be mentioned: cross-linked polyvinylpyrrolidones, particularly crospovidone, modified starches, particularly sodium starch glycolate, modified celluloses, particularly croscarmellose sodium (cross-linked sodium carboxymethylcellulose) and LHPC (low-substituted hydroxypropylcellulose). The disintegrant or superdisintegrant may be present in an amount of from about 2% to about 90%, preferably from about 3% to 60% of the composition.

The valsartan product of these compositions and produced by the methods described herein may be administered to man or animal. The compositions described herein may be administered as pharmaceutical compositions. The valsartan composition may be administered in a therapeutically effective amount to a human or animal in need of such treatment. The term “therapeutically effective amount” as used herein refers to an amount of a pharmaceutical ingredient that is effective to treat, prevent or alleviate the symptoms of a disease. The pharmaceutical compositions of the present invention may be used alone or in combination with other anti-hypertension agents to treat a variety of diseases such as, but not limited to, the treatment of hypertension in children and adults.

Furthermore, compositions of the current invention may include additional active ingredients to the valsartan. Additional active pharmaceutical ingredients include, but are not limited to: analgesics, anti-arrhythmics, anti-bacterials, anti-convulsants, anti-Alzheimer's agents, anti-diabetics, anti-emetics, anti-fungals, anti-histiminics, anti-hyperlipidemics, anti-hyperlipoproteinemics, anti-hypertensives, anti-inflamatory agents, anti-Parkinsonian agents, anti-pulmonary hypertensives, anti-rheumatics, anti-ulceratives, anti-virals, cardiovascular agents, chemotherapy agents, central nervous system sedatives and stimulants, diuretics, gastrointestinal agents, hormones, respiratory agents, skin agents, as well as actives for the treatment of acne, benign prostatic hypertrophy, and irritable bowel syndrome.

The present invention is described in more detail by the following non-limiting examples.

### EXAMPLES

### Example 1

Two spray dried powders were produced on a Mini Spray Dryer (Buchi Labortechnik AG). The first powder was prepared by the solvent only method and contained 47.4% VAL, 47.7% PVP (Plasdone® K-29/32, ISP Corp.), and 4.9% fumed silica (Aerosil, Degussa Corp.) and was spray dried at 5% total solids from methanol which is a solvent for PVP. The second powder was prepared by the solvent/non-solvent method and contained 50% VAL and 50% HPC (Klucel® EF, Hercules, Inc.) and was spray dried at 5% total solids from a methanol/ethyl acetate solution. Methanol is a solvent for HPC while ethyl acetate is a non-solvent.

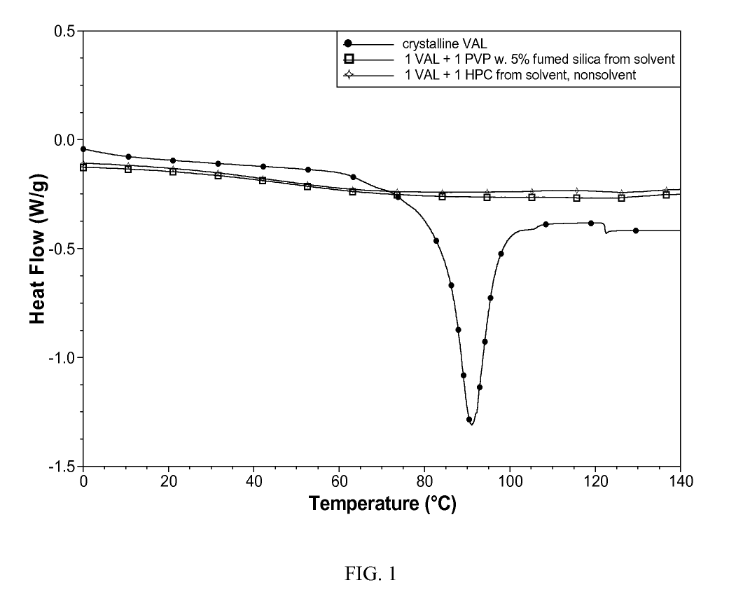

The crystallinity of the two powders was evaluated using DSC (Q1000, TA Instruments). Both powders contained VAL only in the amorphous form, as indicated by a lack of a DSC endothermic (i.e., melting) thermal event (FIG. 1)

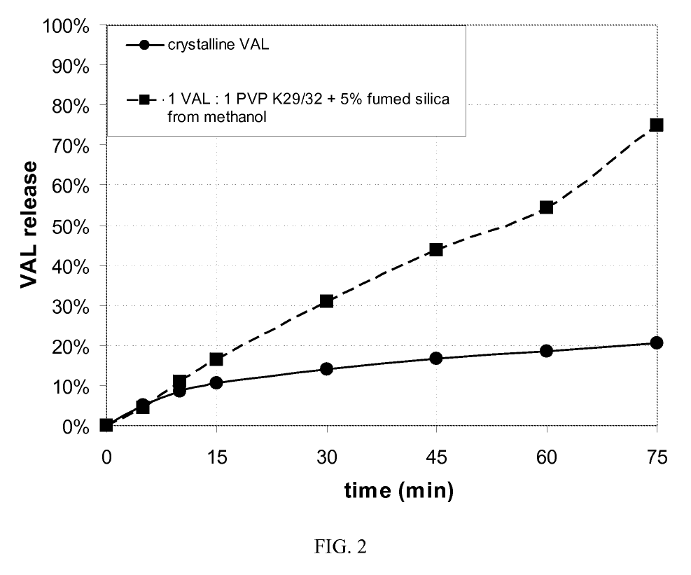

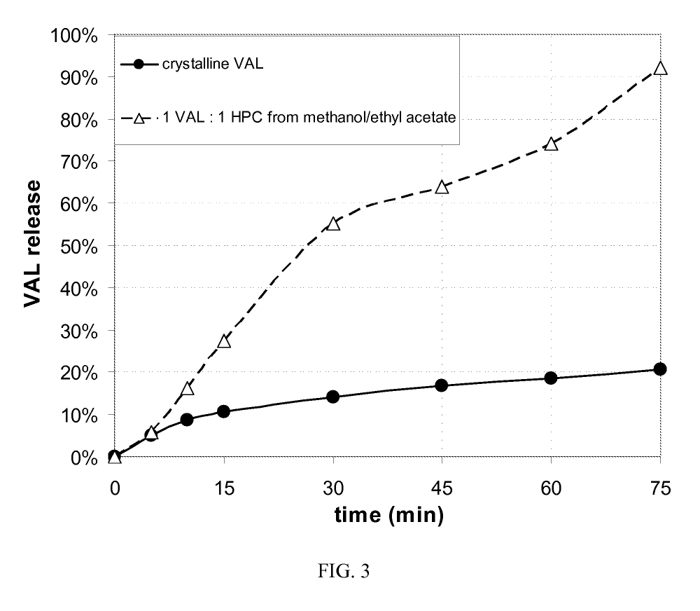

Dissolution properties of the two amorphous spray dried powders and the crystalline drug were measured using a Varian 7010 dissolution bath (apparatus 2, paddles) and a Cary 50 UV spectrophotometer. The paddle speed was 100 rpm for the first 60 minutes, followed by 200 rpm for an additional 15 minutes. USP water was the test medium. Compared to the crystalline form, the amorphous, PVP-based spray dried composition prepared by the solvent only method enhanced VAL release by 45% in the first 15 minutes and by 450% in 75 minutes (FIG. 2). Compared to the crystalline form, the HPC-based composition prepared by the solvent-non-solvent method enhanced VAL release by 145% in the first minutes and by 540% in 75 minutes (FIG. 3).

### Example 2

The relative bioavailability of the PVP based composition of Example 1 was compared to the crystalline form in male Sprague Dawley rat in a parallel study design. An oral, solid dose of 100 mg/kg was administered to the rats in the fasted state. The amorphous VAL composition achieved 40% higher bioavailability, as indicated by area under the twenty-hour hour drug plasma concentration vs. time (AUC₀₋₂₄ₕ) profile of 40 μg h/mL for the crystalline form vs. 56 μg h/mL for the spray dried amorphous form. Results are significant to the 95% confidence level.

### Example 3

Three spray dried powders were produced using a Mini Spray Dryer (Buchi Labortechnik AG). The powders were 1 VAL: 1 polyvinylpyrrolidone (Plasdone® K-12) (ISP) from a blend of dichloromethane/acetone, 1 VAL: 1 polyvinylpyrrolidone-co-vinyl acetate (Plasdone® S-630) (ISP) from a blend of dichloromethane/acetone, and 1 VAL: 1 hydroxypropylmethylcellulose (Pharmacoat® 603) (Shin-Etsu Chemical Co. Ltd.) from a blend of dichloromethane/methanol. While dichloromethane is a solvent for polyvinylpyrrolidone, acetone is a non-solvent, in that it does not swell the polymer molecule (α<1).

All powders contained amorphous VAL, as evidenced by the lack of a melting endotherm as measured by DSC (Q1000, TA Instruments).

### Example 4

Six dispersion blends were prepared using the spray dried powders of Example 3 (Table 1). These blends were prepared combining the spray dried powders with excipients using either a dry blending or a ballmilling method. Dry blended samples were prepared by triturating weighed quantities of spray dried powder and adjuvants using a mortar and pestle. A Retsch® ballmill was used to mill the spray dried powders with adjuvants. Weighed quantities of the spray dried powder and the excipients were milled for 2 hours at 200 rpm using stainless steel balls (25 in count; 9.9 mm ID). All materials were assayed for drug content in order to accurately dose them in a Sprague Dawley male rat model. An oral, solid dose of 50 mg/kg was administered to the rats in a fasted state.

The rat study indicated that both bioenhanced and bioequivalent formulations were produced, depending on the polymer used to make the amorphous solid dispersion and the blended excipients. This analysis is based on the geometric mean ratios of AUC₀₋₂₄ₕ between the amorphous formula and the crystalline VAL control. Geometric mean ratios greater than 1.25 indicate bioenhancement, which was attained by four of the test formulas (Table 1). Geometric mean ratios between 0.8-1.25 indicate bioequivalence, which was attained by two of the test formulas.

TABLE 1
Relative bioavailabilities of amorphous valsartan preparations in a Sprague Dawley rat model.

| | dispersion blends | | |
|---|---|---|---|
| spray dried powder | excipients | method | $\frac{{AUC}_{0 - 24 h} for dispersion}{{AUC}_{0 - 24 h} for control}$ |
| 1 VAL:1 Plasdone ® K-12 | (no excipient blend) | (no excipient blend) | 1.4 |
| | 93% SDD with 7% | PM | 1.4 |
| | sodium bicarbonate | | |
| | 50% SDD with | PM | 1.2 |
| | 15% croscarmellose | | |
| | sodium and 35% | | |
| | microcrystalline cellulose | | |
| | 50% SDD with 25% | BM | 1.3 |
| | croscarmellose sodium | | |
| | and 25% lactose | | |
| | monohydrate | | |
| 1 VAL:1 Plasdone ® S-630 | 50% SDD with 25% | BM | 1.7 |
| | croscarmellose sodium | | |
| | and 25% lactose | | |
| | monohydrate | | |
| 1 VAL:1 HPMC | 50% SDD with 25% | BM | 0.9 |
| | croscarmellose sodium | | |
| | and 25% lactose | | |
| | monohydrate | | |

PM = physical mixture
BM = ballmilled

Changes may be made by persons skilled in the art in the compositions and/or in the steps or the sequence of steps of the method of manufacture described herein without departing from the spirit and scope of the invention as defined in the following claims.

What is claimed is:

1. A composition comprising a solid dispersion of valsartan and a solubility-enhancing polymer wherein said valsartan is substantially amorphous and exhibits enhanced bioavailability compared to a control composition without the solubility-enhancing polymer.

2. The composition of claim 1 wherein said valsartan is completely amorphous.

3. The composition of claim 1 wherein the polymer hydroxypropylcellulose is selected from the group consisting of polyvinylpyrrolidone, hydroxypropylmethylcellulose, hydroxypropylmethylcellulose acetate succinate, hydroxypropylmethylcellulose phthalate and mixtures thereof.

4. The composition of claim 1 wherein the ratio of valsartan to solubility-enhancing polymer is between about 25% valsartan:75% polymer to about 75% valsartan:25% polymer.

5. The composition of claim 1 wherein the composition comprises spray dried particles of valsartan and polymer.

6. The composition of claim 5 wherein the spray dried particles of valsartan and polymer have an average particle size of from about 0.5 μm-500 μm.

7. A pharmaceutical dosage form comprising the composition of claim 1.

8. The pharmaceutical dosage form of claim 7 wherein the dosage form comprises an oral, solid-dosage form.

9. The pharmaceutical dosage form of claim 8 wherein the dosage form provides a maximum plasma concentration for a pharmaceutically active form of valsartan that is at least 1.25 times greater than that of a control composition containing crystalline valsartan.

10. The pharmaceutical dosage form of claim 8 wherein the amorphous valsartan provides an increase in the exposure (AUC₀₋₂₄ₕ) of at least 1.25 times that of a control composition containing crystalline valsartan.

11. A method for providing valsartan to a subject comprising administering to said subject the oral, solid dosage form of claim 8.

12. The method of claim 11 wherein said dosage form is administered to treat hypertension.

13. A method of preparing an valsartan composition comprising:
contacting a quantity of valsartan with a solubility-enhancing polymer in a solvent system comprising a solvent for the polymer, and removing the solvent to form an valsartan-polymer composition wherein the valsartan exhibits enhanced bioavailability.

14. The method of claim 11 wherein the solvent is removed by spray drying the mixture to form particles comprising valsartan.

15. The method of claim 11 wherein the solvent system further comprises a non-solvent for the polymer.

16. The method of claim 13 wherein the solvent and non-solvent are present at a ratio of from about 5% solvent:95% non-solvent to about 95% solvent:5% non-solvent.

17. The method of claim 13 wherein the valsartan exhibiting enhanced bioavailability exhibits faster dissolution, greater extent of dissolution, or both compared to an valsartan composition made without a non-solvent for the polymer.

18. The method of claim 15 wherein the concentration of the polymer in the mixture is from about 1% to about 90%.

19. The method of claim 15 wherein the valsartan in said mixture is almost completely amorphous.

20. A method for preparing a composition comprising amorphous valsartan comprising:
a. providing a mixture comprising valsartan and a solubility-enhancing polymer in a solvent or a blend of a solvent and non-solvent for the solubility-enhancing polymer;
b. distributing the mixture into either droplets or granules, and
c. evaporating the solvent or solvent and non-solvent from the mixture to form a composition comprising particles wherein the particles comprise amorphous valsartan.

21. The method of claim 20 wherein the solubility-enhancing polymer is selected from the group consisting of hydroxypropylmethylcellulose, hydroxypropylcellulose, hydroxypropylmethylcellulose acetate succinate, hydroxypropylmethylcellulose phthalate, polyvinylpyrrolidone and mixtures thereof.

22. The method of claim 20 wherein the particles have an average size of from about 0.5 μm to about 5000 μm.

23. The method of claim 20 wherein the mixture comprises a blend of a solvent and non-solvent for the solubility-enhancing polymer.

24. The method of claim 23 wherein said particles possess less crystalline drug than particles produced from a mixture containing solvent alone.

25. The method of claim 23 wherein the mixture comprises a solubility-enhancing polymer/solvent/non-solvent combination selected from the group consisting of polyvinylpyrrolidone/dichloromethane/acetone, polyvinylpyrrolidone/ethanol/cyclohexane, polyvinylpyrrolidone-co-vinyl acetate/acetone/hexane, and ethylcellulose/acetone/water.
